(19) United States
(12) Patent Application Publication
(10) Pub. No.: US 2023/0104317 A1
(43) Pub. Date: Apr. 6, 2023

(54) Systems and Methods to Identify Genetic Silencers and Applications Thereof

(71) Applicant: The Board of Trustees of the Leland Stanford Junior University, Stanford, CA (US)

(72) Inventors: Baoxu Pang, Stanford, CA (US); Michael P. Snyder, Stanford, CA (US)

(73) Assignee: The Board of Trustees of the Leland Stanford Junior University, Stanford, CA (US)

(21) Appl. No.: 17/759,917

(22) PCT Filed: Feb. 1, 2021
(86) PCT No.: PCT/US21/16078

§ 371 Date: Aug. 1, 2022

Related U.S. Application Data
(60) Provisional application No. 62/968,582, filed on Jan. 31, 2020.

(51) Int. Cl. C12N 15/10; C12N 9/22; C12N 9/90

(52) U.S. Cl. CPC C12N 15/1086; C12N 9/22; C12N 9/90

(57) ABSTRACT

Embodiments herein describe systems and methods to identify genetic silencers. Many embodiments screen one or more DNA fragments to identify sequences that are capable of silencing gene expression. Once identified, silencer elements can be utilized for many applications, including precision medicine.

### STATEMENT REGARDING FEDERALLY SPONSORED RESEARCH OR DEVELOPMENT

This invention was made with Governmental support under Contract No. HG009442 and HG017735 awarded by the National Institutes of Health. The government has certain rights in the invention.

### SEQUENCE LISTING

This application hereby incorporates by reference the material of the electronic Sequence Listing filed concurrently herewith. The material in the electronic Sequence Listing is submitted as a text (.txt) file entitled “06599_SeqList_ST25.txt” created on Jan. 26, 2021, which has a file size of 1 KB, and is herein incorporated by reference in its entirety.

### FIELD OF THE INVENTION

The present invention relates to identifying genetic elements in a genome. More specifically, the present invention relates to systems and methods to identify genetic silencers and utilizing these silencers in various applications.

### BACKGROUND

Less than 2% of the 3 billion base pairs in the human genome codes for proteins. The majority is non-protein-coding, and includes repeat regions, noncoding RNAs, gene introns and other intergenic regions1. Individual laboratories as well as large consortia such as ENCODE (Encyclopedia of DNA Elements) and Roadmap Epigenomics have made enormous contributions to annotating the noncoding genome with epigenetic modifications and transcription factor binding sites. Based on the profiling of epigenetic modifications, the human genome can be categorized into distinct functional units including (but not limited to) enhancers, insulators, promoters, and silencers. However, to date most research has focused on defining enhancers, insulators and promoters. Although silencers are an important class of regulatory elements, thus far, most studies have been performed on identifying and characterizing individual silencer regions, and high-throughput methods have not been described to systematically identify genomic silencers. As such, silencers have been understudied and underappreciated.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a method to identify genetic silencer from a biological source includes obtaining or having obtained a DNA fragment, inserting the DNA fragment into an expression construct comprising a promoter operatively linked with a gene, where the fragment is proximal to the promoter and the gene produces a suicide protein, introducing the expression construct into a biological cell, determining whether the DNA fragment contains a silencer element by inducing toxicity of the suicide protein, and sequencing the DNA fragment within the biological cell to identify a sequence of a silencer element.

In a further embodiment, obtaining or having obtained the DNA fragment includes obtaining or having obtained DNA from a biological source and fragmenting the DNA to a desired size.

In another embodiment, the biological source is selected from the group consisting of animal cells, plant cells, bacteria, fungi, archaea, viruses, viroids, virions, organelles, organoids, tissues, whole organism, and biopsies.

In a still further embodiment, the suicide protein is a fusion protein comprising a binding protein and an apoptotic protein.

In still another embodiment, the suicide protein is a fusion protein comprising FK506 binding protein fused with caspase 9.

In a yet further embodiment, inducing toxicity of the suicide protein involves introducing a dimerizer molecule to the biological cell.

In yet another embodiment, the dimerizer molecule is AP20187.

In a further embodiment again, the expression construct further comprises a selectable marker or a fluorescent marker.

In another embodiment again, the expression construct includes a selectable marker, and wherein the biological cell is grown in the presence of puromycin, hygromycin, neomycin, or bleomycin.

In a further additional embodiment, introducing the expression construct into a biological cell comprises a viral vector transformation, transfection, or electroporation.

In another additional embodiment, the expression cassette is introduced into the biological cell via a viral vector and the viral vector is a lentivirus, a retrovirus, an adenovirus, an adeno-associated virus, a baculovirus, a vaccinia virus, or a herpes simplex virus.

In a still yet further embodiment, the viral vector is transduced at a low multiplicity of infection.

In still yet another embodiment, the method further includes synthesizing a DNA molecule comprising the sequence of the identified genetic silencer.

In a still further embodiment again, the method further includes modifying a genetic silencer in a genome of a second biological cell, wherein the genetic silencer has a matching sequence to the silencer element identified via sequencing.

In still another embodiment again, modifying the genetic silencer is accomplished via CRISPR/Cas9.

In a still further additional embodiment, an expression construct includes a promoter operatively linked to a gene encoding for a suicide protein and a DNA fragment to be screened, wherein the DNA fragment is proximal to the promoter.

In still another additional embodiment, the promoter is a constitutive promoter.

In a yet further embodiment again, the suicide protein is a fusion protein comprising a binding protein and an apoptotic protein.

In yet another embodiment again, the suicide protein is a fusion protein comprising FK506 binding protein fused with caspase 9.

In a yet further additional embodiment, the construct further comprises a selectable marker.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a schematic expression construct or expression cassette of a suicide gene in accordance with various embodiments.

FIG. 2 illustrates a method to identify silencer elements in DNA in accordance with various embodiments.

FIG. 3 illustrates exemplary results from two biological replicate experiments in K562 cells. Each dot represents one tested fragment. The position of the dot indicates the location of each fragment within the respective chromosome. The height of the dot indicates the —log10(FDR) of enrichment of the ReSE fragments after the induction of apoptosis compared with the cells not treated with dimerizer. The cutoff value of FDR is 0.01.

FIG. 4 illustrates a snapshot of distribution of silencers in K562 cells. Chromosome 11 is used as an example.

FIG. 5 illustrates an exemplary distribution of significantly enriched silencer regions from K562 cells in the genome. The pie chart indicates the distribution of silencers in genomic features. The bar plot indicates the distribution of silencers in overlapped annotation features in the genome.

FIG. 6 illustrates luciferase assays to determine the repressive activity of identified silencers from K562 cells. Silencer regions were cloned by PCR from the genomic DNA of K562 cells, and then inserted upstream of the promoter of a luciferase reporter plasmid pGL4.53. 293T cells were used as the control cell line to control for cell-type-dependent repressive activity, and empty pGL4.53 plasmid was used as the control for baseline luciferase activity. The y-axis represents the percentage of luciferase activity compared to pGL4.53 empty plasmids in the respective cells. (n=3 biological independent samples; the bars show the mean value±S.E.M; *P value<0.05, calculated using two-sided Student's t test).

FIG. 7 illustrates exemplary results from two biological replicate experiments in HepG2 cells. Each dot represents one tested fragment. The position of the dot indicates the location of each fragment within the respective chromosome. The height of the dot indicates the —log10(FDR) of enrichment of the ReSE fragments after induction of apoptosis compared with the cells not treated with dimerizer. The cutoff value of FDR is 0.01.

FIG. 8 illustrates a comparison of silencer regions identified from K562 and HepG2 cells. Overlapping was not random as determined by permutation tests (n of testing=20,000, adjusted P value=0.00005).

FIG. 9 illustrates luciferase assays to determine the repressive activity of shared silencers from K562 and HepG2 cells. Silencer regions were cloned by PCR from the genomic DNA of K562 cells into a luciferase reporter plasmid pGL4.53. 293T cells were used as the control cell line and empty pGL4.53 plasmid was used as the control for baseline luciferase activity. The y-axis represents the percentage of luciferase activity compared to pGL4.53 empty plasmids in the respective cells. (n=3 biological independent samples; the bars show the mean value±S.E.M; *P value<0.05, calculated using two-sided Student's t test).

FIG. 10 illustrates top canonical pathways enriched in gene sets containing silencers in K562 cells (CVS, cardiovascular system).

FIG. 11 illustrates top canonical pathways enriched in gene sets containing silencers in HepG2 cells.

FIG. 12 illustrates an exemplary distribution of silencer regions in respective chromatin states in K562 cells. (Txn, transcription; Repressed, Polycomb repressed; lo, low signal; CNV, copy number variation). Colored parts indicate chromatin states that are significantly enriched compared to the library background distribution (P value<0.001, one-sided binomial test).

FIG. 13 illustrates an exemplary association of histone modifications with silencers in K562 cells. The y-axis indicates the significance of the enrichment of histone modifications with silencer fragments. The size of the circle indicates the ratio of silencers covered by the respective histone modification (scale is 0 to 1). The enrichment analysis is based on permutation tests using 20,000 random permutations. The P value was calculated and multiple comparison corrections were computed using the Benjamini-Hochberg procedure. The red line shows the cutoff of the adjusted P value<0.05.

FIG. 14 illustrates an exemplary association of transcription factors with silencers in K562 cells. The y-axis indicates the significance of the enrichment of transcription factors with silencer fragments. The size of the circle indicates the ratio of silencers covered by the respective transcription factor (scale is 0 to 1). The enrichment analysis is based on permutation tests using 20,000 random permutations. The P value was calculated and multiple comparison corrections were computed using the Benjamini-Hochberg procedure. The red line shows the cutoff of the adjusted P value<0.05.

FIGS. 15A-15B illustrates examples of top known motifs present in the silencer regions in K562 cells.

FIG. 15C illustrates an example of a top de novo motif present in the silencer regions identified from K562 cells.

FIG. 16 illustrates an exemplary strategy to use CRISPR/Cas9 to remove silencers from the genome and test their function endogenously in accordance with various embodiments.

FIG. 17 illustrates luciferase assays to determine the repressive activity of silencers from within ABCC2 and ABCC2 genes in K562 cells. 293T cells were used as the control cell line and empty pGL4.53 plasmid was used as the control for baseline luciferase activity. The y-axis represents the percentage of luciferase activity compared to pGL4.53 empty plasm ids in the respective cells. (n=3 biological independent samples; the bars show the mean value±S.E.M.; P value<0.05, calculated using two-sided Student's t test).

FIG. 18 illustrates exemplary results from knocking out silencer from ABCC2 gene using CRISPR/Cas9. PCR result showing the removal of the silencer from the ABCC2 gene of two K562 cell clones, which is the representative result of 2 experiments.

FIG. 19 illustrates exemplary results from knocking silencer from ABCG2 gene using CRISPR/Cas9. PCR results showing the removal of the silencer from the ABCG2 gene of one K562 cell clone, which is the representative result of 2 experiments.

FIG. 20 illustrates ABCC2 gene expression quantified by qPCR. (n=3 biological replicates of the same clones; the bars show the mean value±S.D.; *P value <0.05, calculated using two-sided Student's t test).

FIG. 21 illustrates ABCG2 gene expression quantified by qPCR. (n=3 biological replicates of the same clones; the bars show the mean value±S.D.; *P value <0.05, calculated using two-sided Student's t test).

FIG. 22 illustrates drug resistance promoted by the up-regulation of ABCC2 and ABCG2 genes after silencer removal. (n=3 biological replicates of the same clones; the bars show the mean value±S.D.; *P value<0.05, calculated using two-sided Student's t test; Doxo: doxorubicin; Daun: daunorubicin and Etop: etoposide).

FIG. 23 illustrates exemplary TADs identified by Hi-C data. Arrow indicates the relative location of the silencer.

FIG. 24 illustrates ChIA-PET interactions identified using RAD21 (component of cohesin complex) and CTCF surrounding the ABCC2 gene.

FIG. 25 illustrates exemplary transcription results of genes within different TADs quantified by qPCR. The blue bar indicates genes that do not reside in the same TAD with the ABCC2 gene. The yellow bar indicates genes that reside in the same TAD as the ABCC2 gene. (n=3 biological replicates of the same clones; the bars show the mean value±S.D.; *P value<0.001, calculated using two-sided Student's t test).

FIG. 26 illustrates exemplary results of direct interaction of silencers with promoters of distal genes from 5C analysis in accordance with various embodiments.

FIG. 27 illustrates exemplary results of gene up-regulation by removing promoter-interacting silencers from the endogenous regions. The expression of respective genes with promoter-silencer interactions was quantified by qPCR. (n=3 biological replicates of the same clones; the bars show the mean value ±S.D.; *P value <0.05, calculated using two-sided Student's t test).

### DETAILED DESCRIPTION

Turning now to the drawings and data, embodiments related to identification of genetic silencers and applications of their use are provided. In several embodiments, genetic silencers are identified utilizing fragments of DNA within a screening protocol. In many embodiments, numerous DNA fragments are inserted into an expression cassette comprising a promoter operatively linked with a toxic gene. In a number of embodiments, the expression cassettes containing various DNA fragments and toxic gene are introduced into a cell such that the toxic gene is expressed unless it is silenced by the genetic fragment. In some embodiments, a viral vector system (e.g., lentivirus) is utilized to generate viral vectors with the DNA fragments and toxic gene expression cassette for transduction into a host cell to perform the screen. In many embodiments, genetic silencers are identified by their ability to prevent cellular toxicity.

As noted above, many embodiments are directed to methods of identifying silencer elements present in genomes. Silencer elements, in accordance with many embodiments are segments of DNA capable of preventing transcription. Additional embodiments are further capable of identifying elements capable of repressing or suppressing transcription.

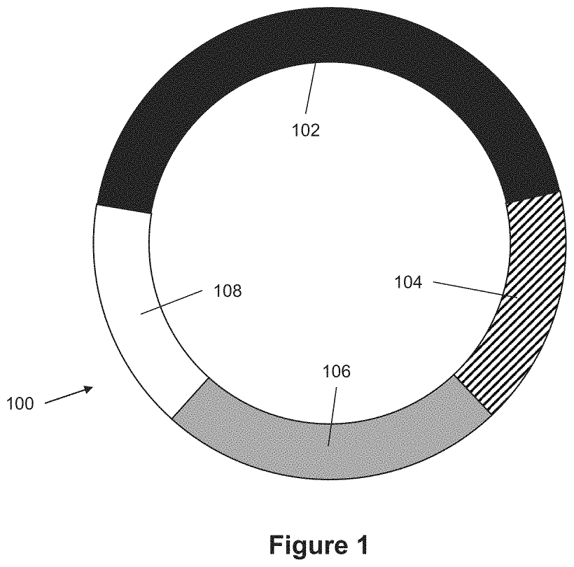

Traditional reporter mechanisms will not work to identify silencer elements, as traditional reporting mechanisms (e.g., GFP) rely on driving expression of reporter genes, while silencer elements actually prevent expression. In light of such challenges, many embodiments incorporate suicide mechanisms or genic constructs designed to drive apoptosis or cellular death upon activation or expression of the suicide protein. As such, silencer elements prevent expression, thus preventing apoptosis. Turning to FIG. 1, various embodiments are directed to constructs to identify silencer elements, such as construct 100. In many embodiments, a construct 100 includes a segment of DNA 102 incorporated into a plasmid or other cassette or construct capable of expression within a cell or tissue. In various embodiments, the DNA is obtained from genomic DNA (gDNA). In certain embodiments, the DNA is enriched for certain segments of DNA, such as exonic DNA, non-coding DNA, and/or DNA associated with particular proteins.

### Constructs to Identify Silencer Elements

In many embodiments, construct 100 further includes a promoter 104 operatively linked to a suicide construct to drive expression of the suicide construct. In many embodiments, the DNA fragment 102 is upstream of the promoter (i.e., in the 5′ direction). The promoter can be any suitable promoter for a particular cell being used for screening (e.g., a mammalian promoter for mammalian cells, bacterial for bacterial cells, etc.). Some embodiments use the EF-la promoter.

For the suicide mechanism, various embodiments utilize an inducible protein for apoptosis. In certain embodiments, the inducible protein is activated using a binding domain that in turn activates a protein in the apoptosis pathway. In some embodiments, the inducible protein is a fusion protein comprising a binding protein 106 and an apoptotic protein 108. In certain embodiments, the binding protein 106 is a FK506 binding protein (FKBP), and the apoptotic protein 108 is caspase 9 (Casp9).

Cells transformed or transfected with suicide protein constructs, such as construct 100 in FIG. 1, express a condition that causes cellular death, unless the suicide protein is silenced. In various embodiments including a fusion protein, such as FKBP-Casp9, the addition of a dimerization molecule activates the apoptotic protein. Specifically, for embodiments of the FKBP-Casp9 fusion, a dimerizer molecule AP20187 binds to FKBP, thus activating Casp9 to induce apoptosis. However, if the gDNA segment 102 contains a silencer element, then suicide protein is not expressed. Thus, apoptosis will not be induced via inclusion or introduction of a dimerizing molecule or other system to activate the apoptosis protein.

Further embodiments of construct 100 include additional genes or elements to assist in transfection, integration, selection, sequencing, amplification, restriction, and/or any other mechanism that may be useful in molecular techniques. For example, numerous embodiments of construct 100 include an expressible marker to indicate and/or select cells that are expressing the marker. In some embodiments, the construct 100 includes a selectable marker. A selectable marker is an expressed gene that promotes resistance to toxins such as (for example) puromycin, hygromycin, neomycin, bleomycin, etc. In some embodiments, the construct 100 includes a fluorescent marker. Fluorescent markers are genes that express a protein that provides fluorescence such as (for example) green fluorescent protein, blue fluorescent protein, red fluorescent protein, yellow fluorescent protein, emerald, turquoise, venus, citrine, cerulean, cherry, tomato, plum, etc. Further embodiments include origins of replication to allow for replication of the construct in bacterial intermediates. One of skill in the art would understand additional elements or features that may be included within a construct for a particular use.

Methods to Identify Silencer Elements

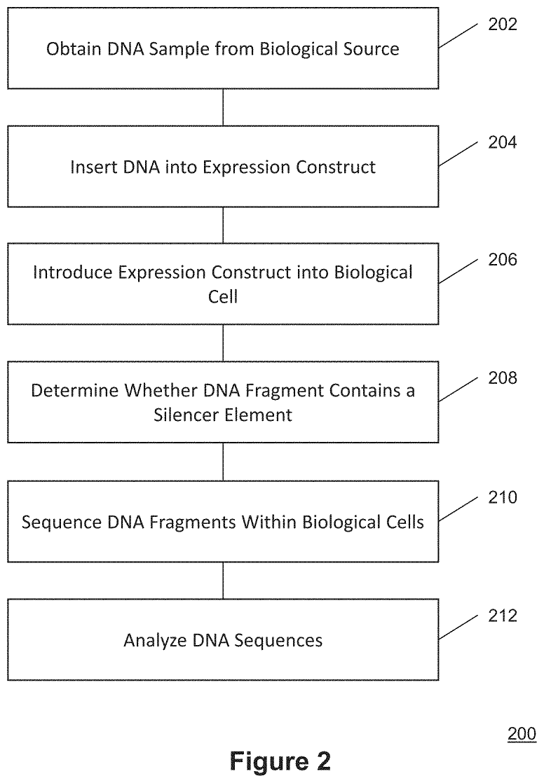

Provided in FIG. 2 is a method 200 to identify genetic silencers. In several embodiments, fragments of DNA are screened to determine their ability to silence gene expression. In many embodiments, the ability of a DNA fragment to silence a toxic gene is used as a primary readout, indicating that the DNA fragment contains a genetic silencer.

In many embodiments, method 200 begins with obtaining (or having obtained) a DNA sample at 202. As noted above the DNA can be gDNA or DNA that is enriched for particular sequences. The DNA can be sourced from any biological source including animal cells, plant cells, bacteria, fungi, archaea, viruses, viroids, virions, organelles, organoids, tissues, whole organism, biopsies, and/or any biological source. Several embodiments are directed to utilizing DNA fragments derived from open chromatin. Accordingly, various methods can be utilized to obtain DNA fragments, including (but not limited to) Formaldehyde-Assisted Isolation of Regulatory Element (FAIRE) (see P. G. Giresi, et al., Genome Research 17, 877-885, (2007), the disclosure of which is incorporated by reference in its entirety). It should be understood, however, that other regions, including (but not limited to) condensed chromatin, can be utilized in various embodiments. In many embodiments, the DNA is fragmented to a particular length or desired size that allows its insertion into a vector or expression cassette. Fragmentation in accordance with various embodiments can occur by many means, including via sonication, pressure shearing, nebulization, restriction digest, acoustic shearing, and/or any other means known in the art.

At 204 of many embodiments, DNA fragments are inserted into an expression cassette or construct comprising a promoter operatively linked with a gene to drive expression of the gene. . In many embodiments, the gene is a toxic gene (e.g., suicide gene or suicide protein). Such constructs are described above in relation to FIG. 1. In some embodiments, constructs are provided in plasmid DNA form, which may facilitate growth and expansion of the expression vector in a bacterial system (e.g., E. coli). In some embodiments, constructs are provided within a viral vector backbone.

In many embodiments, a single fragment is introduced into a single cassette or construct via methods known in the art. In various embodiments, the DNA fragment in each cassette or construct is proximal, or near to, the promoter, such that any regulatory effect of the fragment will affect the promoter. In various embodiments, the DNA fragment is 5′ of the promoter.

Many embodiments introduce the expression construct within a biological cell at 206 to express the toxic gene within the biological cell. Any appropriate means to introduce the expression cassette into the cell can be utilized, including (but not limited to) transfection, viral vector transduction, electroporation, etc. In some embodiments, DNA is transfected into a cell via a lipid transfection reagent (e.g., Thermo Fisher Scientific Lipofectamine) or a chemical transfection reagent (e.g., polyethylenimine (PEI)). In some embodiments, DNA is electroporated into a cell utilizing electrical pulses (e.g., Lonza Nucleofector).

In several embodiments, viral vectors are utilized to transduce the expression construct into a biological cell. Any appropriate viral vector may be utilized, including (but not limited to) lentivirus, retrovirus, adenovirus, adeno-associated virus, baculovirus, vaccinia virus, herpes simplex virus, etc. Viral vector can be prepared by an appropriate means, as necessary by the particular vector used. In many embodiments, an expression construct comprising a toxic gene operatively linked to a promoter and genetic DNA fragment are inserted into a viral vector backbone, which is then packaged into a viral vector. Typically, viral vector helper constructs are utilized to propagate and package viral vectors to yield a viral vector titer. Packaged viral vector yields can be stored or used immediately for transduction. In some embodiments, viral vectors are transduced with low multiplicity of infection (MOI) such that cells are likely to be transduced with a single expression construct having a unique genetic DNA fragment. In some such embodiments, an MOI of 0.01 to 1.0 is utilized. In some embodiments, the MOI is 0.01, 0.05, 0.1, 0.2, 0.3, 0.4, 0.5, 0.6, 0.7, 0.8, 0.9, or 1.0.

In many embodiments, cells with the expression construct are purified and/or selected. In some embodiments, cells are selected via a co-expressed marker, such as a selectable marker or fluorescent marker. In some of these embodiments, cells expressing a fluorescent marker are purified utilizing flow cytometry. In some of these embodiments, cells expressing a selectable marker are purified by growing the cells in the presence of the selection agent (e.g., puromycin, hygromycin, neomycin, bleomycin, etc). In some embodiments, stable cell lines that express the selectable marker are generated by continuously growing the cells in the presence of the selection agent.

Utilizing the transfected/transduced cells, method 200 determines whether the fragment of DNA within the expression construct contains a genetic silencer based upon the ability of a cell to survive due to silenced expression of the toxic gene at 208. In several embodiments, the toxicity of the gene product is induced. In some embodiments, the toxic gene product is activated by dimerization and thus toxicity is induced utilizing a chemical dimerizer such as (for example) AP20187. In many embodiments, cells that survive the toxicity have a high likelihood of having an operable silencer that silences expression of the toxic gene.

At 210, many embodiments determine the sequence of the DNA fragment within the expression construct. Many methods are known in the art to sequence DNA fragments with or without amplification (e.g., via PCR) prior to sequencing. Actual sequencing can be accomplished via any appropriate sequencing platform, including Sanger platforms (e.g., ABI 3730), Illumina platforms, Roche 454 platforms, Pacific BioSciences Platforms, etc.

Further embodiments analyze sequences identified via sequencing at 212 of many embodiments. In a number of embodiments, sequencing results of transfected/transduced cells are compared to sequencing results of control cells to identify genetic regions that are enriched as determined by read counts. In many of these embodiments, enriched regions contain a putative genetic silencer. In some embodiments, a statistical and/or computational model is utilized to determine whether an enriched region is significantly enriched (as compared to control). In some embodiments, a negative binomial model is utilized to determine if an enriched region is significant.

In various embodiments, the analysis includes mapping the putative silencer elements to identify regions that may be enriched for silencer elements; assembling the putative silencer elements to identify larger fragments that may have a greater impact in combination comparative analysis of the putative silencer elements to identify sequence conservation between putative silencer elements across tissues, species, cells, etc.

It should be noted that various features of method 200 are merely illustrative and exemplary. While specific examples of processes for identifying genetic silencers are described above, one of ordinary skill in the art can appreciate that various features described in relation to method 200 may be performed in a different order, simultaneously, multiple times, or omitted for various purposes in accordance with various embodiments. As such, it should be clear that the various steps of the process could be used as appropriate to the requirements of specific applications. Furthermore, any of a variety of processes for identifying genetic silencers appropriate to the requirements of a given application can be utilized in accordance with various embodiments of the invention. Additionally, while the term silencer or silencer element is used, various embodiments are capable of identifying repressor elements that limit gene expression without completely silencing gene expression.

### Applications of Silencer Elements

Once genetic regions are determined to include a silencer element, various applications can be performed. In some embodiments, a genetic silencer is synthesized to be utilized in various expression modifying applications. In some embodiments, a genetic silencer is utilized within a recombinant system to selectively silence genes of interest. Accordingly, in various embodiments, a genetic silencer can be place within proximity to a promoter to reduce expression of a gene driven by the promoter. In some embodiments, a silencer is inserted within a recombinant expression cassette comprising a promoter and operably linked gene. In some embodiments, a silencer is inserted proximal to a promoter within a cellular genome in order to reduce expression of that gene within that cell. Any appropriate means to modify genetic sequences of a cellular genome can be utilized, such as (for example) CRISPR-Cas9 system.

Various embodiments are also directed towards disrupting and/or ablating a silencer within a cellular genome. Accordingly, mutagenesis can be performed on an identified silencer at a particular location, resulting in an inoperable or weakened silencer and increasing expression of at least one nearby gene. This can be particularly useful in treatments of genetic medical disorders involving abnormally low expression of gene, especially haploinsufficiency. Haploinsufficiency arises when one of two alleles of a gene is either unhealthy due to a mutation or deletion and the healthy allele cannot produce enough gene products to compensate for the loss, resulting in a medical disorder. Thus, in numerous embodiments, a silencer proximal to the healthy allele can be disrupted and/or ablated to increase expression the healthy gene and its product.

A number of medical disorders can arise due to haploinsufficiency and thus can be treated by silencer disruption and/or ablation. Medical disorders arising from haploinsufficiency include (but are not limited to) various cancers, 1a21.1 deletion syndrome, 22q11.2 deletion syndrome, CHARGE syndrome, cleidocranial dysotosis, Ehlers-Danlos syndrome, frontotemporal dementia caused by haploinsufficiency of progranulin, DeVivo syndrome, Dravet syndrome, haploinsufficiency of A20, holoprosencephaly caused by haploinsufficiency in the Sonic Hedgehog gene, Holt-Oram syndrome, Marfan syndrome, myelodysplastic syndrome, Phelan-McDermid syndrome, and polydactyly.

### EXEMPLARY EMBODIMENTS

Although the following embodiments provide details on certain embodiments of the inventions, it should be understood that these are only exemplary in nature, and are not intended to limit the scope of the invention.

### EXAMPLE 1

### Computer Simulation of NASBA Kinetics

BACKGROUND: Methods are presented to identify and define the function of silencer regions in a systematic and high-throughput fashion. This method measures the repressive ability of silencer elements (ReSE) by screening for genomic fragments that repress the transcription of an inducible cell death protein.

METHODS:

Cell Culture:

K562 cells were cultured in RPMI 1640 with L-glutamine, 10% FBS and Pen-Strep. HepG2 cells were cultured in DMEM, 10% FBS and Antibiotic-Antimycotic. Cell density and culture conditions were maintained according to the ENCODE Cell Culture Guidelines.

Library Construction:

FAIRE was performed using K562 cells as previously described. (See e.g., Giresi, P. G., et al. FAIRE (Formaldehyde-Assisted Isolation of Regulatory Elements) isolates active regulatory elements from human chromatin. Genome Research 17, 877-885, (2007); the disclosure of which is hereby incorporated by reference in its entirety.) Briefly, 5×10⁷ K562 cells were fixed with a final concentration of 1% formaldehyde for 5 minutes. 2.5 M glycine was added to a final concentration of 125 mM, and cells were incubated for 5 minutes at room temperature while shaking. Cells were then lysed in 5 ml of Lysis Buffer 1 (50 mM HEPES-KOH, pH 7.5, 140 mM NaCl, 1 mM EDTA, 10% glycerol, 0.5% NP-40, 0.25% Triton X-100) and rocked at 4° C. for 10 minutes. The tubes were subsequently centrifuged at 1,300 g for 5 minutes at 4° C. and the supernatant was removed. The pellet was suspended in 5 ml of Lysis Buffer 2 (10 mM Tris-HCl, pH 8.0, 200 mM NaCl, 1 mM EDTA, 0.5 mM EGTA) and rocked at room temperature for 10 minutes, centrifuged at 1,300 g for 5 minutes at 4° C. and the supernatant was removed. The pellet was then suspended in 2 ml of Lysis Buffer 3 (10 mM Tris-HCl, pH 8.0, 100 mM NaCl, 1 mM EDTA, 0.5 mM EGTA, 0.1% Na-Deoxycholate, 0.5% N-lauroylsarcosine). Cells were then sonicated in bioruptor tubes with sonication beads for 16 cycles for 30 seconds each followed by 30 seconds incubation periods at 4° C. The tubes were centrifuged at 3,000 rpm for 5 minutes at 4° C. and an equal volume of phenol/chloroform (phenol, chloroform, and isoamyl alcohol 25:24:1 saturated with 10 mM Tris, pH 8.0, 1 mM EDTA) was added to the lysate and the aqueous phase was separated with phase lock gel. DNA from aqueous phase was then precipitated with ethanol at −80° C. The pelleted DNA was reverse cross-linked and processed according to the Illumina sequencing library preparation protocol. After Illumina adapters were ligated, fragments were size-selected for 200 bp. PCR procedures using Phusion High-Fidelity PCR 2× Master Mix and PCR primer 1.0 and 2.0 for Illumina TruSeq adapters were: 98° C. for 30 s; 12 cycles of (98° C. for 10 s; 65° C. for 30 s; 72° C. for 30 s); and 72° C. for 5 min. Half of the fragments were further processed for next-generation sequencing using Illumina MiSeq platform to confirm the FAIRE process enriched the proper open chromatin regions. The other half was amplified using primers containing additional sequences by PCR for downstream Gibson assembly. Primer sequences for using Phusion High-Fidelity PCR 2× Master were SEQ ID NOs: 1-2. PCR procedures were: 98° C. for 30 s; 8 cycles of (98° C. for 10 s; 65° C. for 30 s; 72° C. for 30 s); and 72° C. for 5 min. These fragments were then gel-purified.

The ReSE screen lentivirus vector pLenti-FKBP-delCasp9-Puro was designed based on plasmids from Addgene (Plasmid #15567 and #52961). EF-1α, a human constitutive promoter was used to drive the expression of FKBP-Casp9, and the UbC promoter was used to drive the expression of a puromycin-resistance gene. It was reasoned that strong silencer activity would have limited effects on the virus packaging and subsequent puromycin selection, as shown in a retrospective experiment that virus titer and the subsequent puromycin selection were not affected by silencer insertions in the screen plasmid. However, it cannot be ruled out that there might exist “super-silencer” fragments that would affect the virus production or puromycin expression. pLenti-FKBP-delCasp9-Puro plasm ids were digested with BsmBI enzyme and gel-purified. The FAIRE fragments were then inserted into the digested plasmids, 15 bp upstream of the EF-1α using Gibson Assembly. The rationale was to identify a class of strong and more general silencers that are able to repress transcription upstream of the constitutive promoter EF-1α. The assembly mix was made using 50 ng of insert DNA, 50 ng of digested plasmids, and 10 μl of 2× Gibson Assembly Master Mix to produce a final volume of 20 μl. The assembly mix was incubated at 50° C. for 60 min. Then 2 μl of the mix was electroporated into 25 μl of Endura electrocompetent cells to test the transformation efficiency. The electroporation was scaled to reach approximately 160,000 colonies which were plated on 4 245-mm Petri dishes with 100 μg/ml carbenicillin. Colonies were then scraped and plasmid DNA extracted using Qiagen Maxiprep Kit.

Lentivirus Production and Infection:

293T cells were grown in 5 T175 flasks at 50% confluency before transfection. For each flask of 293T cells grown in 25 ml of fresh medium. 15 μg of library plasmids, 10 μg of psPAX2, 5 μg of pCMV-VSV-G and 90 μl of X-tremeGENE 9 DNA Transfection Reagent were mixed in 1 ml serum-free medium and used for transfection. Fresh medium was added the next day after transfection. Media supernatant containing virus particles was collected from the 2nd and 3rd day after transfection, pooled and further concentrated using Lenti-X according to the manufacturer's protocol. Virus titer was then determined by making serial (10-3 to 10-10) dilutions of 4 μl of frozen virus supernatant in media containing 8 μg/ml of polybrene to infect 293T cells. Two days after infection, cells were selected with 2 μg/ml puromycin for an additional 7 days. The virus titer was then calculated based on the survival colonies and the related dilution. K562 cells and HepG2 cells were then infected with the same virus library at MOI 0.5 by spin infection. For spin infection, 3×10⁶ cells in each well of a 12-well plate were infected in 1 ml medium containing 8 μg/ml of polybrene. In total, 4 plates were used for each infection to analyze a total of 1.5×10⁸ cells. Two days after infection, cells were selected by 2 mg/ml puromycin for another 5 days. For each biological replicate experiment of both K562 and HepG2 cells, the infection was repeated and cells were infected with lentivirus from the same pool of virus containing the same library content.

Silencer Screen:

After puromycin selection, 3.5×10⁸ K562 cells were frozen as non-treated control. A separate aliquot of 3.5×10⁸ cells were treated with 1 nM of AP20187 for 18 h to induce apoptosis. Then dead cells were removed with Dead Cell Removal Kit from Miltenyi Biotec. In the screen of K562 cells, we retrieved 45.6% of live cells compared to the original input cell number, after 18 h of AP20187 treatment. If cell growth of the live cells during this 18-h period is also considered, the real survival rate should be around 30.4% (considering the normal doubling time of live K562 cells is 24 hours). In addition, there are also some other scenarios, for example, although cells with virus infection survived puromycin selection (as the expression of puromycin-resistance gene is under another independent promoter UbC promoter), the expression of FKBP-Casp9 was silenced by other machinery within the cells; Or cells were still in the early stage of apoptosis, and were not removed by the live cell isolation method. These could be the reason of higher survival rates. Many such false positive regions were removed during biological repeat experiments. Live cells were further grown for another 5 days. Genomic DNA from 3.5×10⁸ cells of non-treated control cells or post apoptosis-induction cells was isolated using QIAamp DNA Blood Maxi Kit, with 2 columns per treatment. For the K562 cell differentiation test, the same batches of cells that were analyzed as biological replicates were recovered from cells frozen in 10% DMSO. Cells were then differentiated with 10 nM PMA (phorbol 12-myristate 13-acetate) for 2 days. Cells were divided into differentiated non-treated control cells and the other half of cells were treated with 1nM AP20187 for 18 h to induce apoptosis. Dead cells were cleared as described previously. For HepG2 cells, the experiment procedures were similar except that dead cells were removed by removing the media, since HepG2 cells are adherent cells and live cells remained attached to the tissue culture flasks.

Library Sequencing and Analyses:

Genomic DNA containing the ReSE lentivirus inserts was amplified by PCR using Illumina PCR primer 1.0 and 2.0. For each 100 μl PCR reaction, 10 μg of genomic DNA, 20 μl of 5× Phusion HF Buffer, 2 μl of 10 mM dNTP, 2.5 μl of Phusion polymerase, and 5 μl of 25 μM 1.0 primer and 5 μl of 25 μM 2.0 primer were used. For each treatment sample, 16 reactions were prepared and pooled. PCR procedures were: 98° C. for 30 s; 20 cycles of (98° C. for 10 s; 65° C. for 30 s; 72° C. for 30 s); and 72° C. for 5 min. PCR products were then size-selected and purified. Final products were sequenced by IIlumina MiSeq or Hiseq4000 platform. Sequence reads were aligned using Bowtie to hg19. Approximately 100,000 regions of the 177,000 regions within the library were estimated to be well covered in the screen. A GFF file was made from aligned reads pooled from all experiments. Then read counts (quality 30) were calculated using HTSeq74. Final enrichment was calculated by MAGeCK, with two biological replicates for each condition. Briefly, read counts derived from HTSeq of different samples were first median-normalized to adjust for the effect of library sizes and read count distributions. Then the variance of read counts was estimated by sharing information across features, and a negative binomial (NB) model was used to test whether fragment abundance differs significantly between post apoptosis-induction replicates and control replicates. P values were calculated from the NB model using a modified robust ranking aggregation algorithm30. FDR was then computed from the empirical permutation P values using the Benjamini-Hochberg procedure. As fold enrichments are only semi-quantitative, fragments with an FDR lower than 0.01 were considered as significant hits for downstream analyses, and the list of silencers was sorted based on FDR value from low to high.

Luciferase Assay:

Candidate silencer sequences were amplified with primers containing a homologous arm using PCR from the genomic DNA of K562 cells. These fragments were then inserted in front of the PGK promoter of the luciferase plasmid pGL4.53 (Promega) using Gibson assembly. Cells were then co-transfected with the pRL-CMV Renilla reporter vector and the pGL4.53 vector with the silencer sequence inserted. The luciferase assay was performed using the Dual-Glo Luciferase Assay Kit from Promega according to the manufacturer's protocol. Original luciferase plasmid without any insertion was used as the control. All luciferase assays were from 3 independent transfections done on different days.

Pathway Analyses:

Proximal genes around silencers were defined as 1) the presence of silencers only in the promoter regions (10 kb surrounding transcription starting sites [TSS]); or 2) the presence of silencers in both promoter regions (1 kb surrounding TSS) and gene bodies. Pathway analyses were performed using proximal genes with Ingenuity Pathway Analysis (IPA).

CRISPR/Cas9-Guided Silencer Knock-Out:

Guide RNAs targeting the 5-prime and 3-prime ends of the silencer were designed using crispr.mit.edu. The guide RNA sequence was cloned into the PX459V2 plasmid containing the guide RNA scaffold and Cas9 sequence. Two CRISPR/Cas9 plasmids targeting both the 5-prime and 3-prime ends of the silencer were co-transfected into the cells. Cells were then selected for successful transfection using puromycin. Single clones of cells were picked and verified using PCR and Sanger sequencing. Gene expression of target genes was quantified using qPCR, and normalized to the expression of the housekeeping gene GAPDH.

Downstream Informatic Analyses:

Genomic annotation of silencer regions was analyzed using the R packages ChIPseeker and CEAS. Motif analyses were performed using Cistrome. For Motif analyses, only the silencers outside the promoter regions were used for the analyses to reduce bias from motif-rich promoter regions, though we did not observe major differences using all silencers or only the silencers outside of the promoter regions for motif analyses. Region intersections, comparisons, binomial test and other downstream analyses were calculated using R, ChiPpeakAnno, Galaxy or Cistrome. Enrichment of chromatin states was calculated using a one-sided binomial test against the whole ReSE library as the background. ChIP-seq data from ENCODE and dbSNP147 data were downloaded from the UCSC Genome Browser Database with the hg19 genome assembly. Association of histone modifications and transcription factor binding regions with silencers was calculated using the R package ChIPseeker. The enrichment analysis is based on permutation tests using 20,000 random permutations. The P value was then calculated and multiple comparison corrections were computed using the Benjamini-Hochberg procedure for the adjusted P value. For comparing silencers with capture Hi-C data from human primary blood cells or 5C data from K562 cells, silencer regions identified from the ReSE screen in K562 cells were intersected with the distal regions that interacted with the promoter regions from the respective studies. Hi-C, ChIA-PET and 5C data of K562 cells were used and visualized using genome browsers.

RESULTS:

Identification of Silencers:

To systematically discover silencer regions in the human genome, a high-throughput ReSE lentiviral screen system was developed. In this system, genomic regions are cloned upstream of the EF-1α promoter that drives the expression of a modified caspase 9 fused to an FK506 binding protein (FKBP-Casp9). Upon the addition of a dimerizer molecule AP20187, the expressed caspase 9 is activated to induce apoptosis. The system was designed such that if silencers are inserted, they will repress the transcription of the FKBP-Casp9 gene in the cells, and these cells will not undergo apoptosis. Surviving cells are then expanded and candidate inserts sequenced and mapped to the genome. This method allows for the systematic identification of silencer regions.

Presently, it is difficult to screen the entire human genome with small genomic fragments in a lentiviral assay. Therefore, an enrichment strategy was used. It has been shown that 94.4% of the combined transcription factor ChIP-seq peaks from the ENCODE project fall within accessible regions. Many of these transcription factors are associated with transcriptionally repressive activities. Therefore, it was expected that at least some silencers might lie in accessible chromatin regions, as shown for the regulation of the CD4 gene. These silencers would likely harbor regulatory proteins rather than simply be regions that are globally repressed through general heterochromatin mechanisms.

Accessible chromatin regions enriched by FAIRE (Formaldehyde-Assisted Isolation of Regulatory Elements) from chronic myeloid leukemia K562 cells were isolated to construct the ReSE screen library. Briefly, 200-bp accessible chromatin regions prepared from K562 cells were cloned into the ReSE lentiviral plasmids, and a library of more than 177,000 independent regions (covering 1% of the human genome) was constructed and used as the screening library. The library was transduced into K562 cells in two independent replicate experiments, and AP20187 added to induce apoptosis. The surviving cells were grown and the inserts were sequenced before and after selection. The screen has considerable background cell survival during the initial puromycin selection and the subsequent apoptosis induction. Therefore, the fold enrichments are variable due to this and the low read counts (see Methods). Nonetheless, the results from the replicate experiments correlated, although only a small percentage of the potential fragments were consistently enriched between replicates when fold-change was considered.

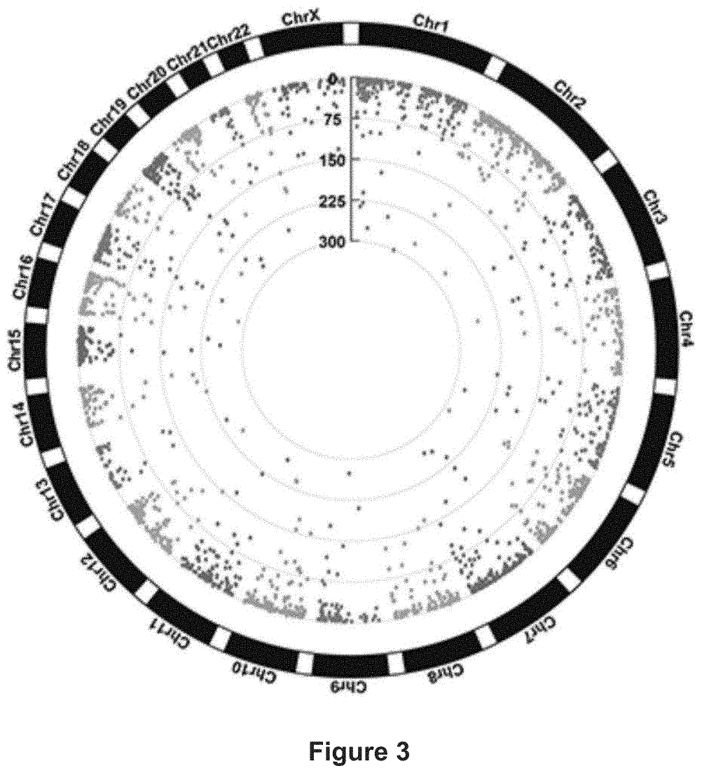

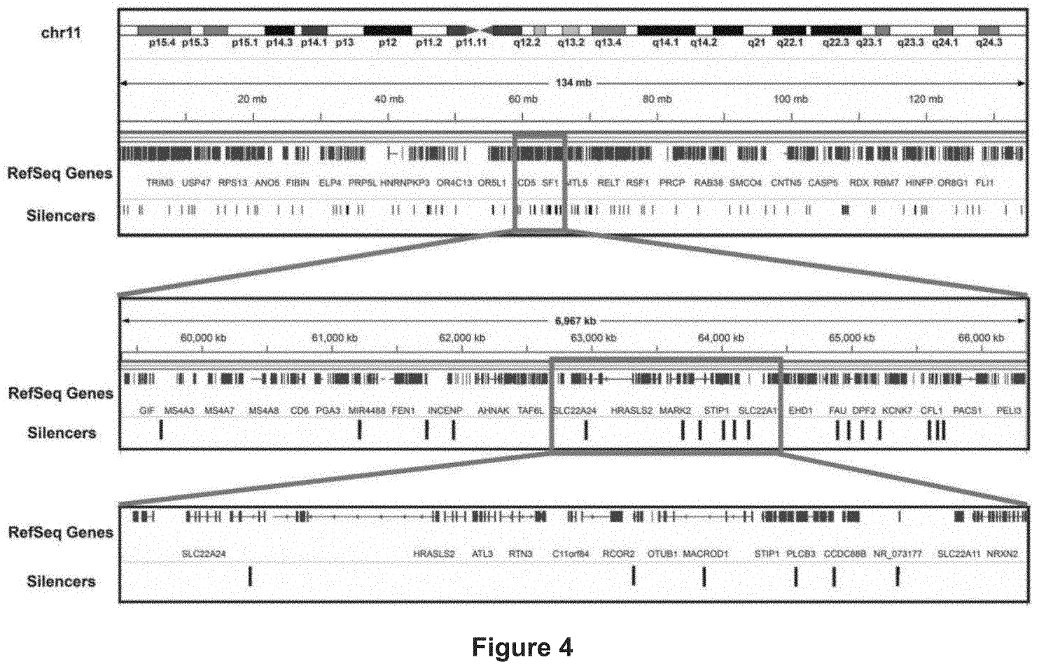

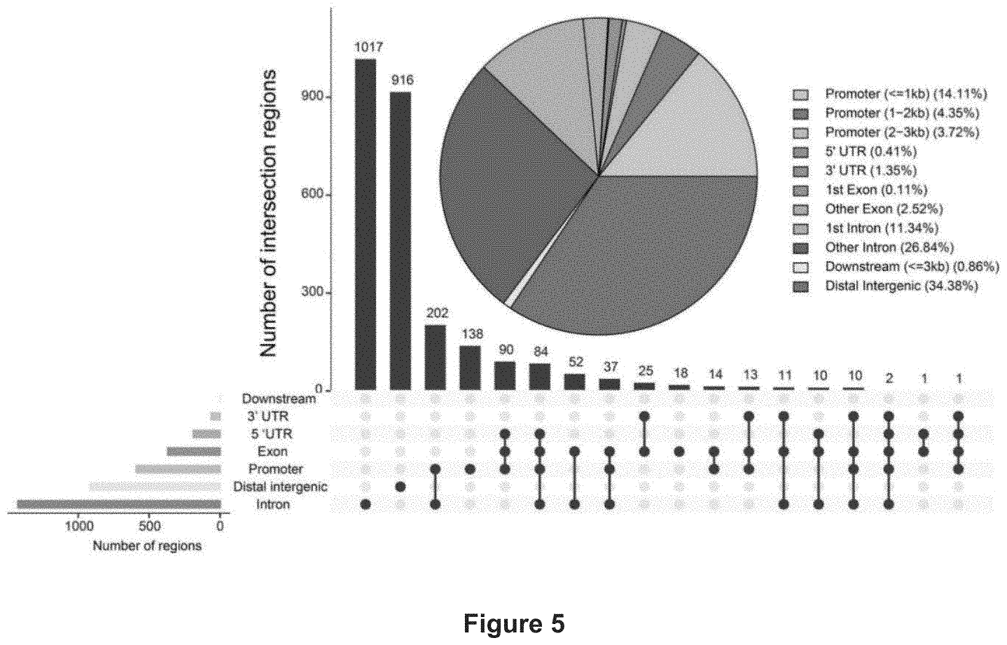

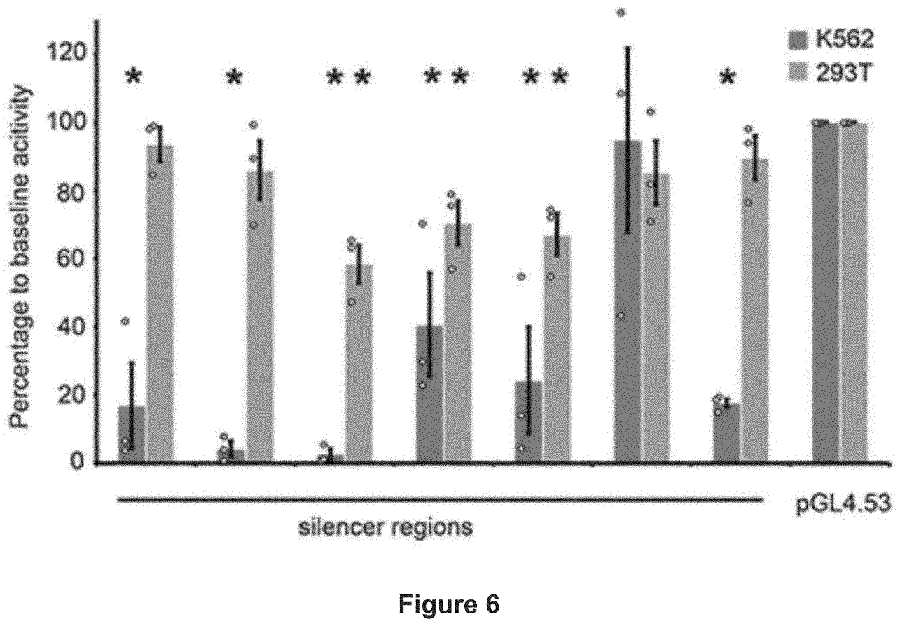

To reliably identify significantly enriched silencers, an algorithm based on a negative binomial (NB) model was adapted, as used previously in CRISPR screen and other RNA-seq differential analyses. This led to the identification of 2,664 potential silencer regions with an FDR cutoff of 0.01 in K562 cells (FIGS. 3-4). The majority of these potential silencer regions were in promoter regions, introns and intergenic regions (FIG. 5). To validate the transcriptional repressive activity of the identified silencer regions, seven regions identified from the screen with the lowest FDR were cloned upstream of a luciferase plasmid with a PGK promoter. Very strong repressive activity was observed from the silencer fragments in K562 cells in 6 of 7 cases (FIG. 6). In addition, testing of five silencers from the bottom of our silencer list revealed that three showed significant repressive activity, suggesting that the high threshold used for calling positives (FDR 0.01) was adequate. The majority of randomly selected regions (eight of nine) from the library did not show repressive ability. These data indicate that most fragments identified in our screen are silencers, and in addition suggest that the activity of most silencers was not limited to the specific promoter used in the initial screen (i.e. the EF-1α promoter).

To test if the silencers can repress their native endogenous genes, three silencer regions from FIG. 6 were deleted from their native loci. Silencer regions located in the intron regions of genes HRH1, SYNE2 and CDH23 were deleted using paired CRISPR guide RNAs that targeted both sides of the individual silencer region. K562 silencer-knockout clones were isolated, and significant up-regulation of the respective genes was observed in each case. These data indicate that silencers identified in the ReSE screen repress the transcription of their nearby endogenous genes.

ReSE Identifies Tissue-Specific and Conserved Silencers:

Since large-scale analysis of silencers has not been performed previously, it was desired to determine if they were common across cell types or whether they function in a tissue-specific manner, similar to enhancers. The same screening library was used to test if a different pool of silencers was enriched during differentiation of K562 cells; the rationale was that if most silencers are common across cell types we would isolate many of the same DNA sequences found in the K562 screen. If the silencers are cell-type-specific, the overlap should be modest. K562 cells were treated with PMA to induce megakaryocytic differentiation. Repeating the ReSE screen in these PMA-treated cells identified a different set of 1,245 silencers compared to those identified in the original K562 cells. This result suggests that silencers may function in a tissue-specific manner.

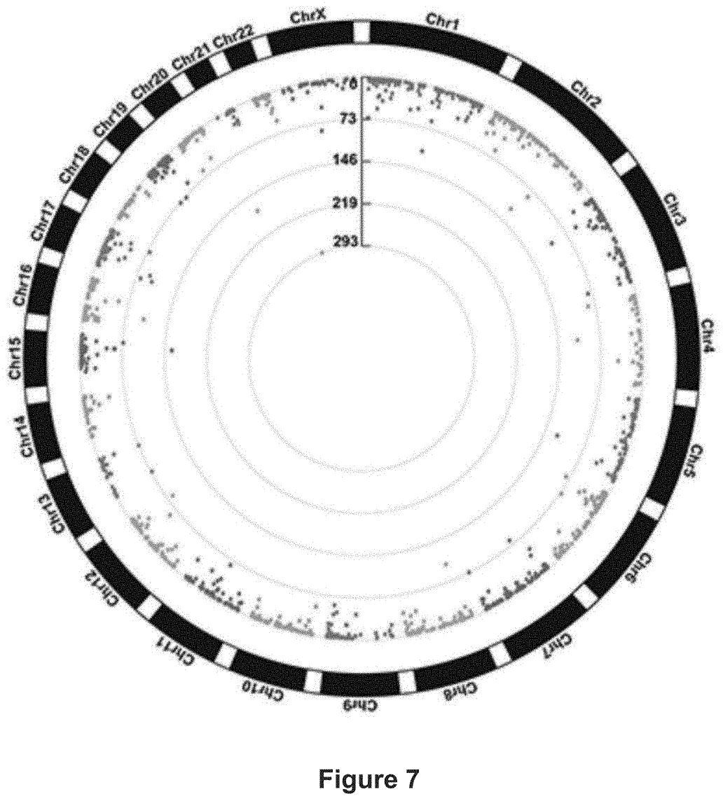

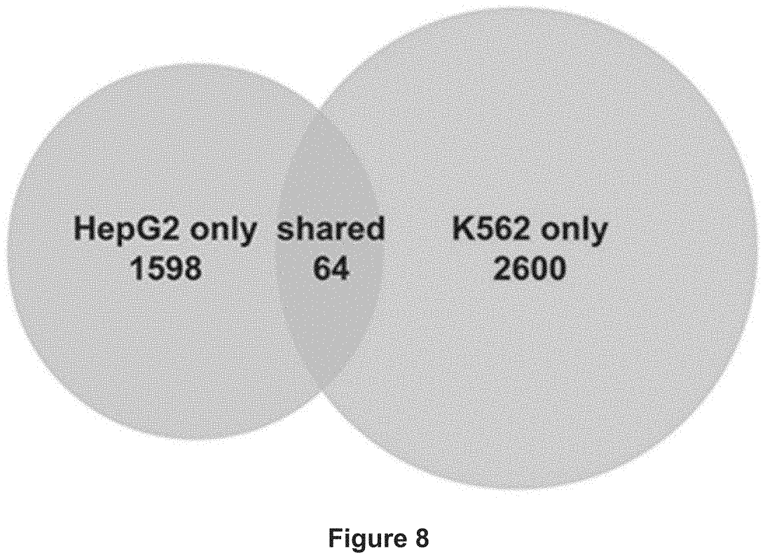

To further test this observation, the ReSE screen was repeated on HepG2 cells that are of hepatocyte origin using the same ReSE library made from the K562 FAIRE enrichment. Again, the rationale was that if the two cell types shared common silencers there would be substantial overlap in the silencers from both cell types. Two independent biological ReSE screens of HepG2 cells led to the identification of 1,662 potential silencer regions with FDR of 0.01 (FIG. 7). Although these silencer regions shared a similar overall genomic distribution with K562 cells, only a small fraction (less than 2% of the total) of the silencer regions was shared between these two cell lines, indicating that the majority of the silencers that we identified, similar to enhancers, may exert their function in a tissue-specific fashion (FIG. 8). However, as a very stringent FDR cutoff was applied to identify silencers in the respective cell lines, it may underestimate the percentage of conserved silencers among different tissues. Nonetheless, the data suggest that many silencers may be tissue-specific.

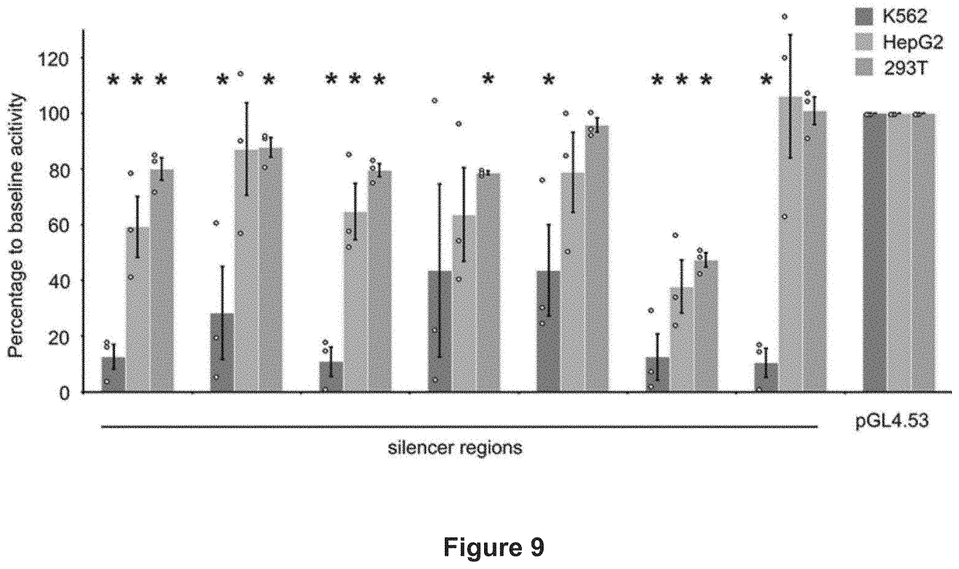

Next, it was directly investigated whether the small percentage of the shared silencers found in K562 and HeG2 cells may be ubiquitous silencers and act in different cell types. To examine this possibility, 7 of the silencer regions shared by both K562 and HepG2 screens were tested using the luciferase assay and 3 were found to be repressive in both cell types (FIG. 9). In addition, repression activity was also observed for 5 out of the 7 common regions tested in 293T cells, suggesting these regions may be cell-type-independent silencers. It should be noted that silencers were called using a stringent algorithm within individual cell lines and the false negative rate may be higher when comparing silencers among cell lines; some top ranked silencers from HepG2 also showed some repressive activity in K562 cells. It is noted that the false discovery rate is higher in HepG2 cells, presumably because the screen library was made from FAIRE regions of K562 cells and we may not have identified the strongest silencers in HepG2 cells. Overall, the current data suggest that the majority of silencers may be cell-type-specific with a small number that operates in two or more cell lines (FIG. 6).

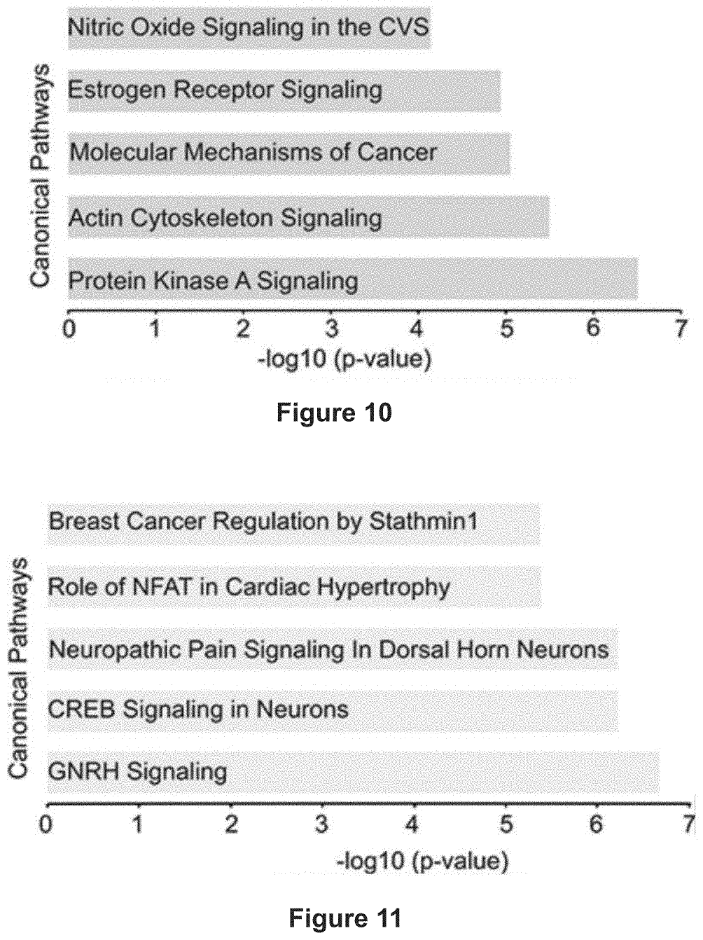

Since the majority of silencers identified by ReSE screen may function in a tissue-specific manner, it was tested if silencers associate with genes in unique pathways. Pathway analyses using Ingenuity Pathway Analysis (IPA) revealed unique pathways with strong confidence (i.e. lower P value) for the proximal genes associated with silencers identified from the different cell types. Two different methods were employed to identify proximal genes that may be regulated by silencers in K562 and HepG2 cells: 1) the presence of silencers only in the promoter regions (10 kb surrounding transcription starting sites [TSS]); 2) the presence of silencers in both promoter regions (1 kb surrounding TSS that is more stringent definition) and gene bodies, since many silencers were enriched in the intron regions (FIGS. 10-11). When the presence of silencers in both promoters and gene bodies are considered, in K562 cells, protein kinase A signaling and actin cytoskeleton signaling pathways were among the top pathways enriched for silencer associated genes (FIG. 10). Since K562 cells are a myeloid cell line growing in suspension culture, it is likely that the actin cytoskeleton signaling pathway is repressed or poised for activation as compared to adherent cells. In contrast, neuronal pathways and cardiac pathways were the top genes that lay near silencers isolated from HepG2 cells (FIG. 11). Such pathways are also expected to be repressed, since HepG2 cells were derived from a hepatic cell lineage.

Silencers Consist of Unique Genetic and Epigenetic Signatures:

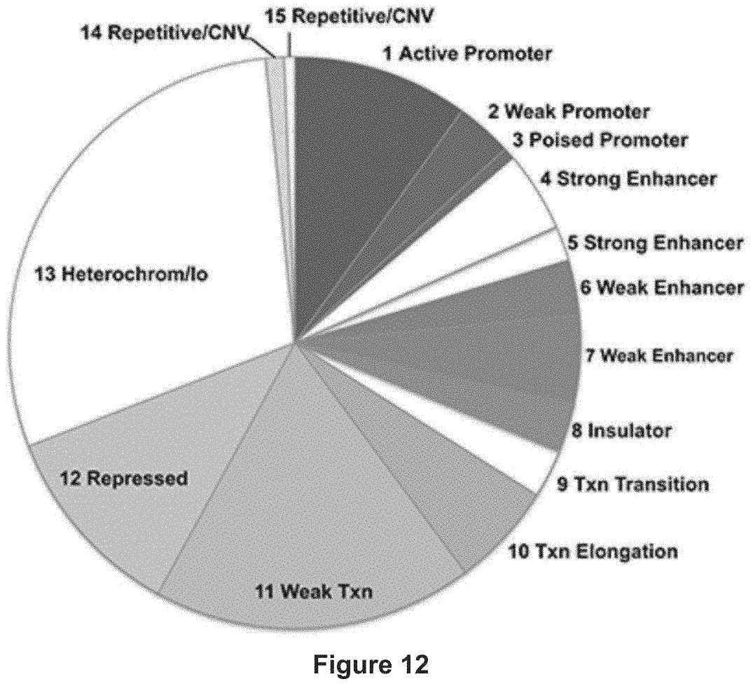

Functional regulatory elements are usually present in defined chromatin states. For instance, enhancer regions often are marked by modifications such as H3K4me1 and H3K27ac10. To determine the chromatin states of silencer regions, ReSE identified, the recovered regions from K562 and HepG2 were first classified based on the ENCODE chromatin definitions. More than one quarter of the silencers is enriched in the weak transcription chromatin state (P value<2.2×10⁻¹⁶, one-sided binomial test using the screen library as the background) or repressed state (P value=3.325×10⁻⁵, one-sided binomial test) (FIG. 12). This indicates that silencers may be associated with specific chromatin modifications that might be necessary to exert their silencing function.

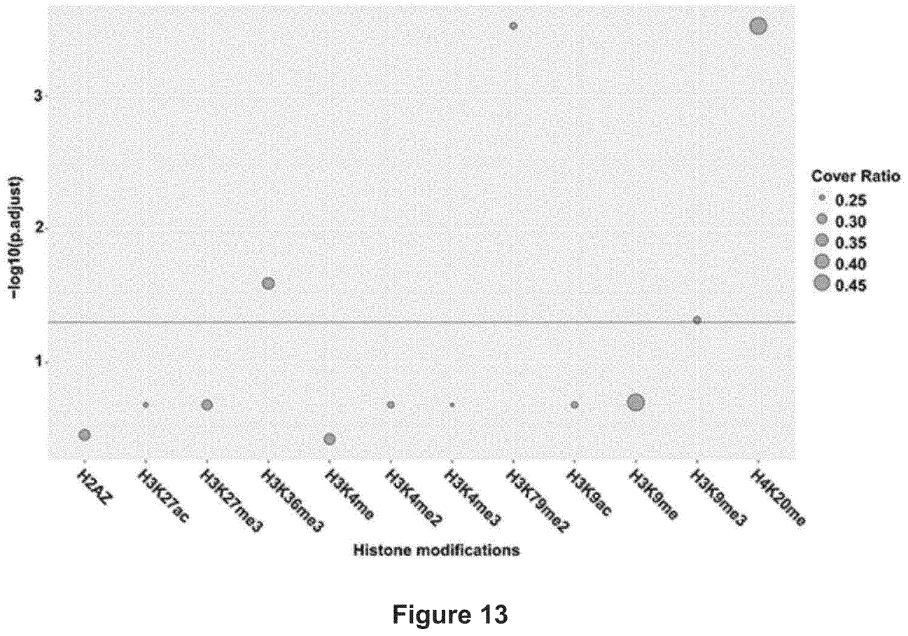

To further examine the epigenetic marks and transcription factors that may be enriched in the silencer regions, a permutation-based test was performed to associate silencer regions with available datasets from the ENCODE project. When histone marks were analyzed, H4K20me modified chromatin was significantly co-associated with silencers from both K562 and HepG2 cells (FIG. 13). The role of H4K20me modification is still complex as both transcriptional repression and activation have been associated with this mark. Interestingly, different heterochromatin histone marks, each associated with gene inactivation, were found to associate with silencers isolated from K562 (H3K9me3; FIG. 13) and HepG2 cells (H3K27me3). This difference may be due to the fact that the initial silencer screen library was constructed from K562 cells, and these silencers may represent a different pool in HepG2 cells. In addition to histone marks related to silencing, H3K36me3 and H3K79me2, both active histone marks, were also significantly associated with silencers. It has been suggested that when combined with other histone marks, active marks like H3K36me3 can be found in heterochromatin regions. This finding is also consistent with the observation that the identified silencers are enriched in weak transcription chromatin states (FIG. 12). It is noted that the selected silencer regions that were targeted for knockout in K562 cells all showed the presence of H4K20me modification further indicating in these representative silencer regions that this mark may be associated with gene silencing (FIG. 13). In addition, these silencers also showed overlap with H3K27me3 or H3K9me3, indicating either a weak transcription chromatin state or a repressed state (FIG. 12).

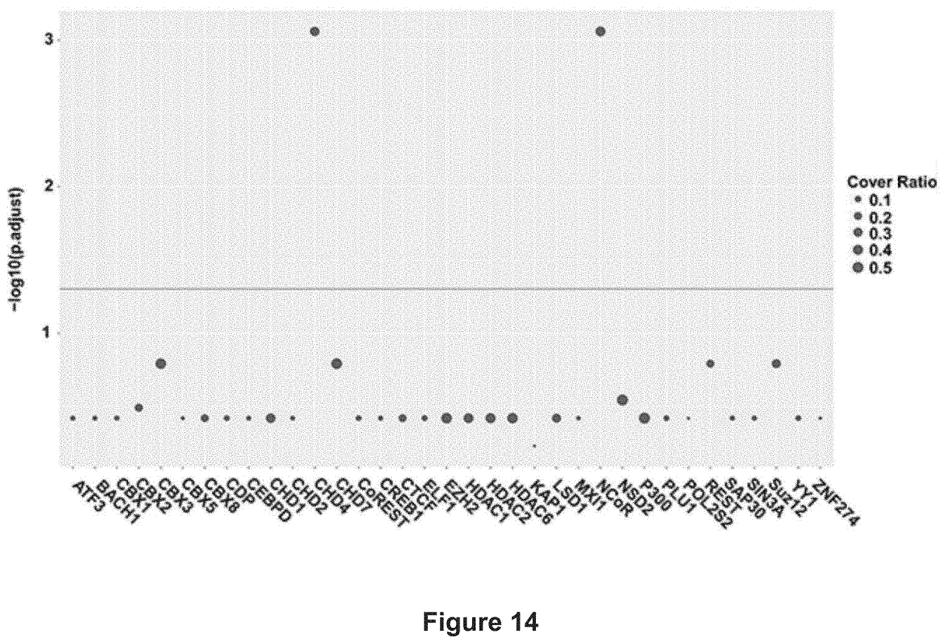

Regulatory proteins that participate in repression might be enriched in silencers. Therefore, a permutation-based co-association test was performed between silencers and regions bound by transcription factors in both K562 and HepG2 cells. In K562 cells, CHD4 and NCoR were significantly enriched in silencer regions (adjusted P value=0.0008; FIG. 14). A different set of transcription factors, e.g. EZH2 and REST, were enriched in HepG2 cells. This likely reflects the cell-type specificity of silencers, as different cell types presumably use different proteins to regulate silencers. While it is likely that different cell types may employ different proteins for silencing, it is also possible that other unidentified silencer proteins are common to different cell types. It is worth noting that among the transcription factors tested, KAP1 and ZNF274 were not associated with silencers identified using our open chromatin screen. This is likely because they are primarily associated with inaccessible chromatin, which is less likely enriched in the screen library. As controls, p300, which is usually associated with transcription activation or Pol2S2 (phosphorylation of Serine 2 on CTD of RNA polymerase II), which is usually associated with active transcription , were also not enriched with silencers.

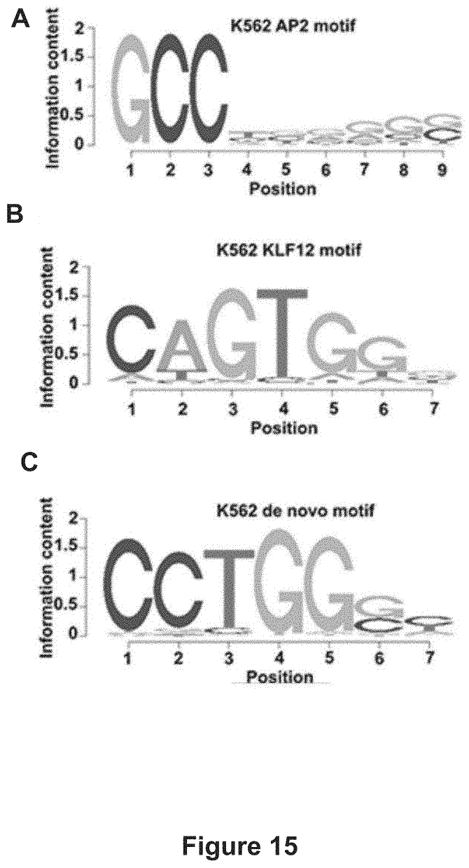

To identify other potential novel factors that may recognize the silencer regions identified by the ReSE screen, motif analyses were performed using SeqPos. The top known motif identified in both K562 and HepG2 cells was the AP2 binding domain (FIG. 15A; with P values=10⁻³⁵⁸ and 10⁻²²⁵ respectively). The role of AP2 family members in transcription repression has been shown previously. However, a general repressive activity of AP2 in different tissues has not been established. Furthermore, the motif of another known transcription repressor KLF12 was also enriched in K562 cells (FIG. 15B). In addition to motifs with known binding factors, many de novo motifs were also identified in both K562 (FIG. 15C) and HepG2 cells. These motifs are GC-rich, similar to AP2 motifs. These data indicate that there may be unique factors that function in silencer regions that are yet to be identified.

Silencers Regulate Proximal Endogenous Genes to Promote Chemoresistance:

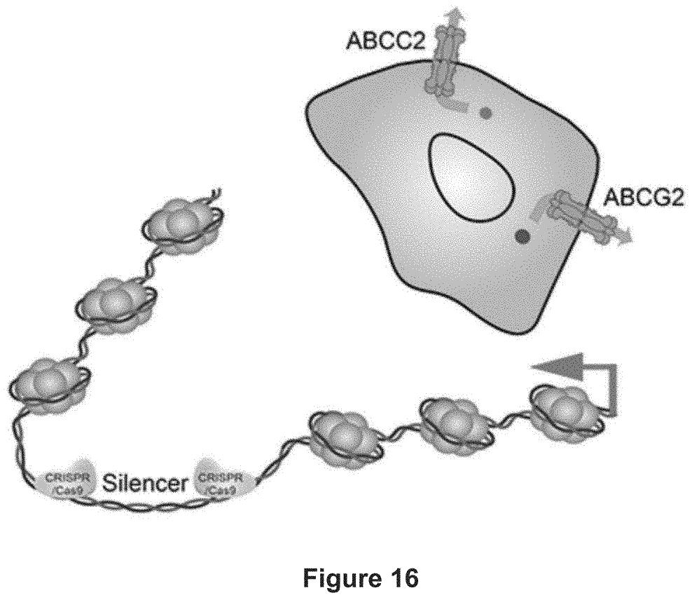

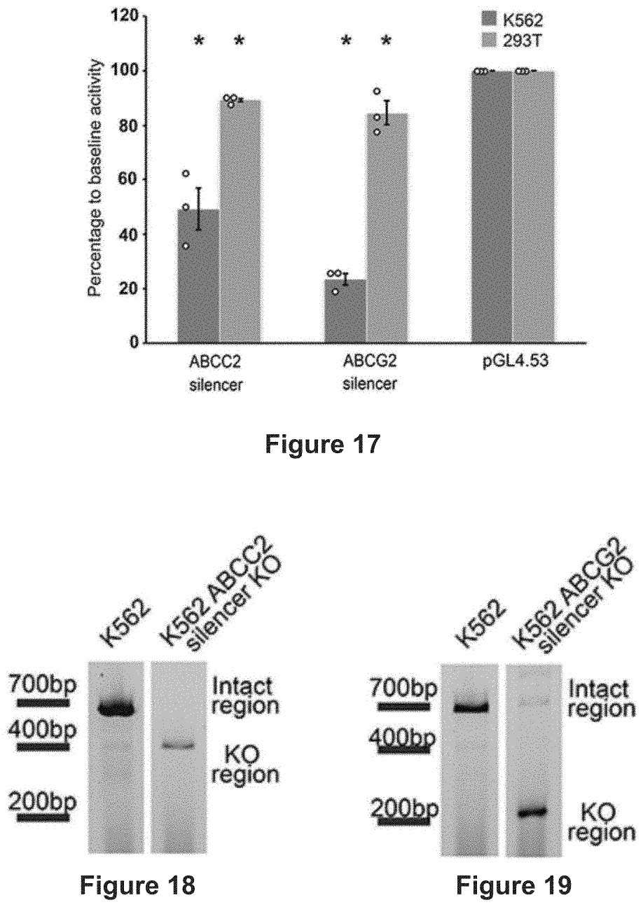

It was next examined if the silencer regions identified by the ReSE screen have direct biological effects. Pathway analyses based on genes that harbor silencers both in the promoter and gene body regions (FIG. 10) revealed that silencers were enriched in genes for an ABC drug transporter pathway in K562 cells (-log(P value)=2.671). Within the intron regions of two drug transporter genes ABCC2 (on chromosome 10) and ABCG2 (on chromosome 4) there exist two potential silencers. It is possible that these silencers affect the transcription of these genes and thus participate in response to drug treatment (FIG. 16). Luciferase assays showed that these silencer regions have repressive activity and repressed the transcription of the pGL4.53 reporter by 50% (ABCC2 locus silencer) and 80% (ABCG2 locus silencer) (FIG. 17).

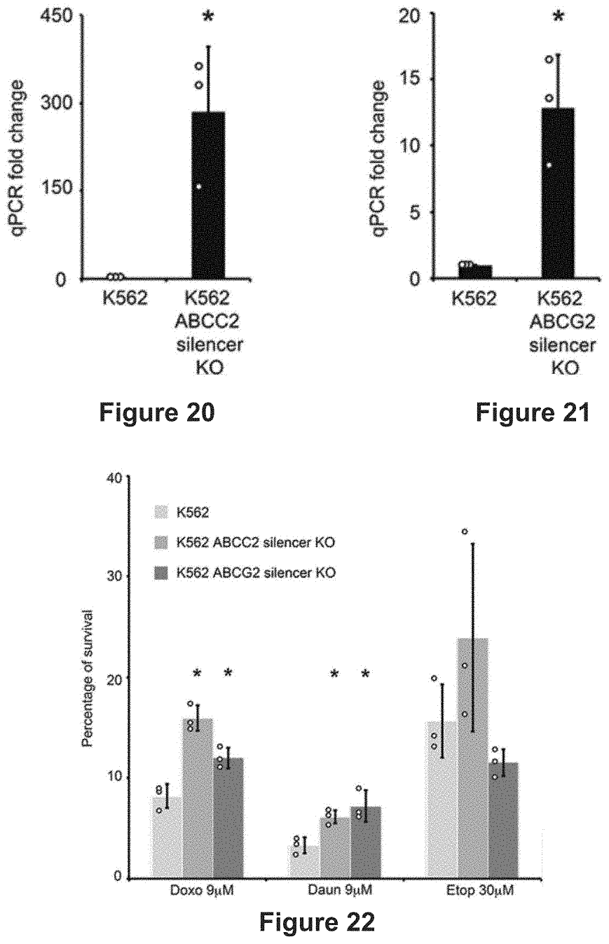

The silencers in the ABCC2 and ABCG2 loci were targeted with flanking CRISPR guide RNAs to delete the regions from the genome (FIGS. 16), and K562 cell clones with the complete knockout of the silencer within ABCC2 (FIG. 18) and ABCG2 (FIG. 19) were derived. Transcription of the ABCC2 and ABCG2 genes was significantly up-regulated in these clones, and such up-regulation was unrelated to puromycin selection since control knockout clones did not show this strong increase (FIG. 20-21). In contrast, knocking out the same silencer regions did not induce significant up-regulation of ABCC2 gene in 293T cells or only modestly up-regulated the ABCG2 gene in 293T cells, in accordance with the luciferase assay in FIG. 16, further indicating that silencers function in a tissue-specific manner. As a result of the transcriptional up-regulation of drug transporters, these knockout clones are both more resistant to chemotherapeutic drug treatments compared to the parental cell line (FIG. 22), suggesting that silencer regions may affect drug sensitivity.

When the local epigenetic modifications were examined, the silencer region in the ABCC2 gene was marked with H2A.Z and H3K27ac, whereas the silencer region in ABCG2 overlapped with H3K27me3 and the H4K20me modification resided nearby. The latter mark is consistent with the results presented in FIG. 13. In addition, the gene bodies of both ABCC2 and ABCG2 were largely covered by H3K27me3 modification, indicating a repressed state of these genes. These two silencer regions were not covered by the H4K20me mark (although the silencer in the ABCG2 locus showed H4K20me in close proximity).

Silencers Regulate Transcription in the 3D Genome:

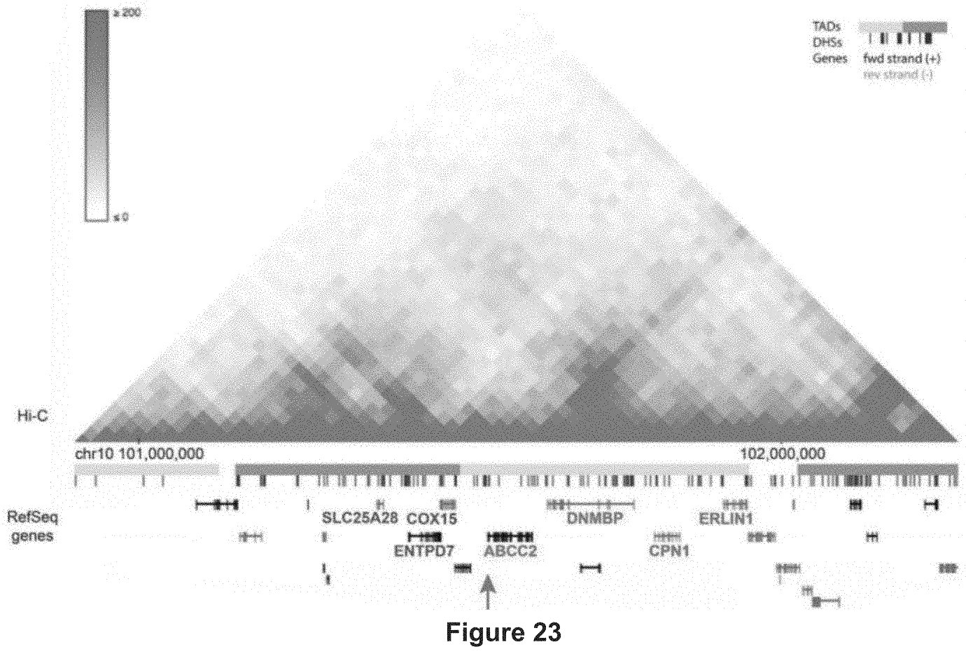

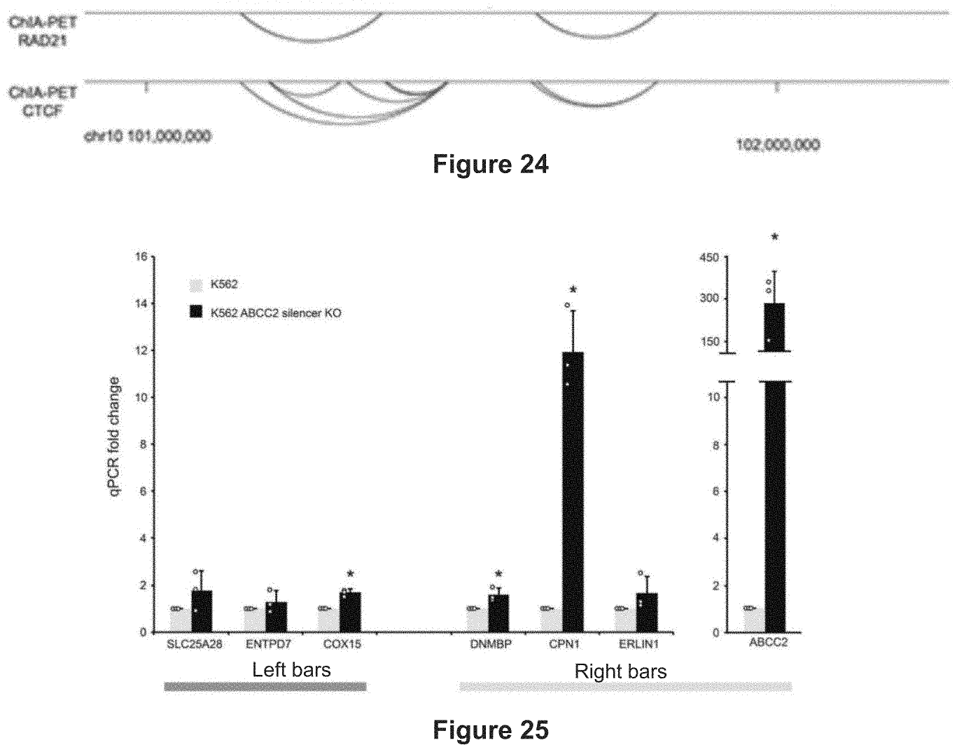

Cis-regulatory elements often regulate not only a single gene, but a group of genes within a topologically associating domain (TAD). To test if this occurs for the ReSE-identified silencers, Hi-C data from K562 cells were integrated to define the different domains surrounding the silencer within the ABCC2 gene (FIG. 23). Hi-C data indicate that one TAD contains the silencer within the ABCC2 gene in K562 cells (FIG. 23). Furthermore, in this TAD a chromatin loop connects both the ABCC2 and CPN1 genes (FIG. 24), as defined by ChIA-PET assays targeting RAD21 and CTCF54. This result raises the possibility that the silencer within the ABCC2 gene may also regulate the CPN1 gene.

Therefore, transcriptional changes of genes from two topologically distinct domains were tested between control and knockout cell lines using qPCR assays. We found that similar to enhancers, silencers also acted on genes within the same chromatin-loop domain, as the CPN1 gene was significantly up-regulated in the K562 ABCC2 silencer KO cell line (12-fold, FIG. 25, “right bars”), in which the ABCC2 gene was also strongly up-regulated (300-fold; FIGS. 19 and 25). Strong gene up-regulation was confined to only these two genes within the TAD (FIG. 23, domain containing ABCC2 gene), in contrast to the genes located in a nearby distinct TAD (FIG. 25, “left bars”).

In order to globally identify distal genes that may be regulated by the silencers, capture Hi-C data that profiled interactions with 31,253 promoter regions from human primary blood cells were integrated with silencers identified from K562 cells. Since K562 cells resemble a common myeloid progenitor origin that is similar to many of the cell types in blood and can be differentiated into many of these cell types, the whole-blood data should contain a lot of these regulatory regions. Silencer regions identified by ReSE in K562 cells interacted with approximately 4,000 promoter regions (permutation test adjusted P value=4.99×10⁻⁵, n=20,000), suggesting that ReSE silencers can directly interact with many promoter regions.

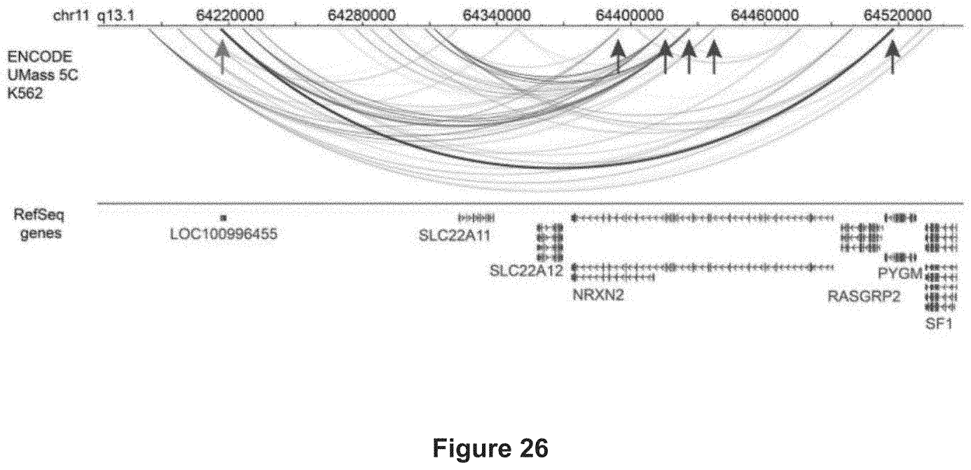

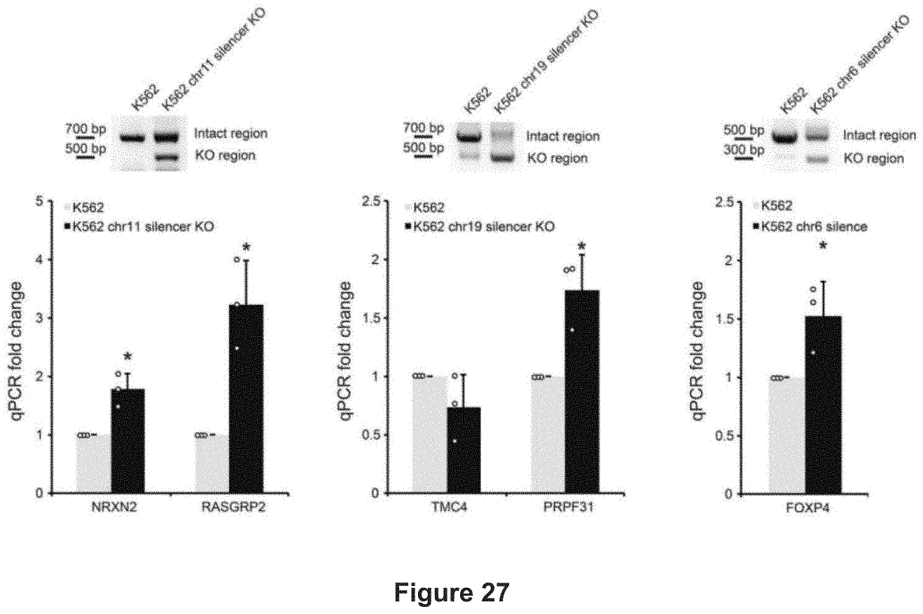

To directly test the effect of promoter-silencer interactions in K562 cells, chromosome conformation capture carbon copy (5C) data from ENCODE that reported long-range interactions between promoter regions and distal elements was examined. However, these 5C experiments only targeted 1% of the genome. 5C data was interacted with the silencer regions identified in K562 cells and found five genes that directly interact with silencer regions (FIG. 26). Among these interactions, three genes NRXN2, TMC4 and FOXP4 showed extremely low expression (FPKM<2) based on RNA-seq data in K562 cells (FIG. 26) and an additional gene RASGRP2 gene showed low expression (FPKM<10) (FIG. 26). When these silencers were removed individually with flanking CRISPR guide RNAs in K562 cells, 4 out of 5 genes were up-regulated significantly in the respective clones (FIG. 27). These data further support the suggestion that silencers may also interact with and regulate distal genes.

CONCLUSIONS:

Although only approximately 2% of the human genome contains coding sequences that can be translated into proteins, many noncoding regions contain unique sequences that can be recognized by chromatin modifiers and transcription factors, as candidate regulatory elements. Systematic analysis of promoters and enhancers has been performed previously, however a global analysis of silencers has not been described. In our study, a robust screening system, ReSE, was developed to systematically identify silencer elements in the human genome. ReSE utilizes a lentiviral system to test the ability of candidate genomic fragments to repress the caspase-based “kill switch” for the enrichment of potential silencers. In principle, other plasmid-based reporter assays normally used for assessing enhancers and promoters could also be used to evaluate silencer activity. However, these systems rely on RNA-seq and therefore may be better suited to evaluate activation rather than repression. Despite the fact that less genomic regions are individually assayed in the lentiviral system compared to other plasmid-based reporter assays, the ReSE lentiviral method can be used to directly select for regions of interest, and in addition it can overcome the plasmid-transfection-related systematic errors that have been realized recently. Nonetheless, akin to other genome-wide screen systems that are intrinsically noisy, ReSE is also limited by false positive and negative discovery. Therefore, multiple biological replicates are recommended for the screen to increase the statistical power. Although silencer regions may exist in different genomic regions with distinct chromatin structures, to prioritize the testing regions in the human genome, we analyzed open chromatin regions, as these regions are accessible for regulatory factors and might directly exert repressive functions rather than passively be silenced through repressive heterochromatin. The ReSE screen reliably identified silencer fragments in different cell lineages. The results suggest that many of the silencers that we identified may function in a tissue-specific manner. Nonetheless, it is possible that a large and common pool of shared silencers exists in different tissues, as the current ReSE screen library was derived only from FAIRE regions of K562 cells and silencers were identified using a stringent cutoff. These data indicate that silencers may play an important role in development and regulate tissue differentiation. Unique motifs are present in the silencers that could potentially be recognized by specific factors to exert repressive functions.

Although the majority of the silencers may be present in the transcriptionally inactive states, presumably some accessible DNA still exists in these chromatin regions to be isolated using FAIRE. Consistent with this interpretation, silencers were found in the vicinity of genes that are poised for responses or repressed during differentiation. Silencers may possess a unique combination of histone signatures (FIG. 13). Such chromatin states may be the result of the recognition of silencers by repressive transcription factors. Since the current ReSE screen was focused on accessible chromatin regions with a fixed testing size (around 200 bp), the resulting signatures, such as histone modifications, transcription factor associations and sequence motifs, may also be biased towards a particular class of silencers using this library. It is possible that there are many different transcription factors recognizing their respective sub-clusters of silencers within distinct cell types (FIG. 14). Future genome-wide analysis of silencers is required to provide a clearer picture of all possible silencing signatures. Systematic identification of silencers in this way helps to further our understanding of the relationship between repressed chromatin states and gene silencing.

Thus far, investigations of drug responses or disease progression often focus on the coding regions of genes, or known regulatory regions such as promoters and enhancers. For instance, a recent CRISPR activation screen targeting promoter regions led to the identification of ABCG2 as an important drug-resistant player in K562 cells. It was found that deleting silencers within drug transporter genes ABCC2 and ABCG2 also led to the up-regulation of these genes and chemotherapeutic drug resistance, suggesting silencer-mediated transcription repression may be another layer of regulation contributing to important medical phenotypes. Thus, it is expected that phenotype-associated genetic variants in silencers may affect drug responses, disease initiation and progression, and be considered as candidates for precision medicine. Furthermore, many diseases are caused by haploinsufficiency or insufficient gene expression. This can be effectively rescued by the newly developed CRISPR/dCas9-mediated activation technology, either by targeting the promoter or enhancer regions of the relevant genes. However, unlike the CRISPR/Cas9-mediated genomic editing/correction that requires only transient expression of the CRISPR/Cas9, the activation system requires constant expression of the CRISPR/dCas9. Therefore, such regulation is often reversible, which may not be ideal for future applications in human diseases. As shown in the data, genomic editing of the silencer regions can lead to gene up-regulation. Therefore, inactivating silencer regions could be complementary to CRISPR/dCas9-mediated activation system to treat many diseases. As such, systematic identification of silencers in the genome using the ReSE screen may not only provide insights into the biology of the genome, but also assist in personalized medicine.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A method to identify genetic silencer from a biological source comprising:
obtaining or having obtained a DNA fragment;
inserting the DNA fragment into an expression construct comprising a promoter operatively linked with a gene,
wherein the fragment is proximal to the promoter, and
wherein the gene produces a suicide protein;
introducing the expression construct into a biological cell;
determining whether the DNA fragment contains a silencer element by inducing toxicity of the suicide protein; and
sequencing the DNA fragment within the biological cell to identify a sequence of a silencer element.

2. The method of claim 1, wherein obtaining or having obtained the DNA fragment comprises:
obtaining or having obtained DNA from a biological source; and
fragmenting the DNA to a desired size.

3. The method of claim 2, wherein the biological source is selected from the group consisting of animal cells, plant cells, bacteria, fungi, archaea, viruses, viroids, virions, organelles, organoids, tissues, whole organism, and biopsies.

4. The method of claim 1, wherein the suicide protein is a fusion protein comprising a binding protein and an apoptotic protein.

5. The method of claim 1, wherein the suicide protein is a fusion protein comprising FK506 binding protein fused with caspase 9.

6. The method of claim 1, wherein inducing toxicity of the suicide protein involves introducing a dimerizer molecule to the biological cell.

7. The method of claim 6, wherein the dimerizer molecule is AP20187.

8. The method of claim 1, wherein the expression construct further comprises a selectable marker or a fluorescent marker.

9. The method of claim 1, wherein the expression construct includes a selectable marker, and wherein the biological cell is grown in the presence of puromycin, hygromycin, neomycin, or bleomycin.

10. The method of claim 1, wherein introducing the expression construct into a biological cell comprises a viral vector transformation, transfection, or electroporation.

11. The method of claim 1, wherein the expression cassette is introduced into the biological cell via a viral vector and the viral vector is a lentivirus, a retrovirus, an adenovirus, an adeno-associated virus, a baculovirus, a vaccinia virus, or a herpes simplex virus.

12. The method of claim 11, wherein the viral vector is transduced at a low multiplicity of infection.

13. The method of claim 1, further comprising synthesizing a DNA molecule comprising the sequence of the identified genetic silencer.

14. The method of claim 1, further comprising modifying a genetic silencer in a genome of a second biological cell, wherein the genetic silencer has a matching sequence to the silencer element identified via sequencing.

15. The method of claim 14, wherein modifying the genetic silencer is accomplished via CRISPR/Cas9.

16. An expression construct comprising:
a promoter operatively linked to a gene encoding for a suicide protein; and
a DNA fragment to be screened, wherein the DNA fragment is proximal to the promoter.

17. The expression construct of claim 16, wherein the promoter is a constitutive promoter.

18. The expression construct of claim 16, wherein the suicide protein is a fusion protein comprising a binding protein and an apoptotic protein.

19. The expression construct of claim 16, the suicide protein is a fusion protein comprising FK506 binding protein fused with caspase 9.

20. The expression construct of claim 16, wherein the construct further comprises a selectable marker.
